(19) United States
(12) Patent Application Publication
(10) Pub. No.: US 2020/0013487 A1
(43) Pub. Date: Jan. 9, 2020

(54) Drug Repurposing Hypothesis Generation Using Clinical Drug-Drug Interaction Information

(71) Applicants: International Business Machines Corporation, Armonk, NY (US); Teva Pharmaceutical Industries, Ltd., Petach-Tikva (IL)

(72) Inventors: Heng Luo, Yorktown Heights, NY (US); Ping Zhang, White Plains, NY (US)

(21) Appl. No.: 16/026,433

(22) Filed: Jul. 3, 2018

(51) Int. Cl. G16H 10/20; G16H 70/40; G06F 17/50

(52) U.S. Cl. CPC G16H 10/20; G06F 2217/16; G06F 17/5009; G16H 70/40

(57) ABSTRACT

Embodiments of the present systems and methods may provide techniques for systematic drug repositioning that fully leverages drug-drug interactions. In embodiments, the present systems and methods may generate a drug similarity metric based on DDI data and make drug repositioning predictions by leveraging the new similarity metric. For example, in an embodiment, a computer-implemented method for conducting a drug repositioning trial may comprise receiving data relating to drug-drug interactions, generating drug interaction vectors for each drug in the received data, generating a logistic regression model to generate drug repositioning hypotheses, generating at least one drug repositioning hypothesis using the logistic regression model, and conducting a drug repositioning trial using the generated drug repositioning hypothesis.

### BACKGROUND

The present invention relates to techniques for generation of drug repurposing hypothesis using clinical drug-drug interaction information.

Conventionally, pharmaceutical drug development has been inefficient, with high expenditure but low productivity. Drug repositioning, the process of finding additional indications, such as diseases, etc., that may be treated with existing drugs, presents a promising avenue for identifying better and safer treatments without the full cost or time required for de novo drug development. Candidates for repositioning are usually either market drugs or drugs that have been discontinued in clinical trials for reasons other than safety concerns. Because the safety profiles of these drugs are known, clinical trials for alternative indications are cheaper, potentially faster and carry less risk than de novo drug development. Any newly identified indications can be quickly evaluated from phase II clinical trials. For example, drug repositioning may reduce drug discovery and development time from 10-17 years to potentially 3-12 years. Therefore, it is not surprising that in recent years, new indications, new formulations, and new combinations of previously marketed products accounted for more than 30% of the new medicines that reach their first markets.

Drug repositioning has drawn widespread attention from the pharmaceutical industry, government agencies, and academic institutions. However, current successes in drug repositioning have primarily been the result of serendipitous events based on ad hoc clinical observation, unfocused screening, and “happy accidents”.

Conventional systematic methods for drug repositioning may include the application of phenotypic screens by testing compounds with biomedical and cellular assays. However, this method also requires additional wet bench work of developing appropriate screening assays for each disease being investigated, and it thus remains challenging in terms of cost and efficiency. Big data analytics for both drugs and diseases may provide an opportunity to uncover novel statistical associations between drugs and diseases in a scalable manner. Conventional computational methods have been developed in this direction, including: (1) matching drug indications by their disease-specific response profiles based on Connectivity Map (CMap) data; (2) predicting novel associations between drugs and diseases by the “Guilt by Association” (GBA) approach; (3) utilizing structural features of compounds/proteins to predict new targets or indications, such as molecular docking, and quantitative structure-activity relationship (QSAR) modeling; (4) identifying associations between drugs and diseases in genetic activities, such as genome-wide association study (GWAS), pathway profiles, and transcriptional responses; (5) constructing drug networks and using network neighbors to infer novel drug uses based on phenotypic profiles, such as side effects, and gene expression. However, these conventional approached do not adequately deal with drug-drug interactions (DDIs), as a more complicated clinical format of drug side effects, for drug repositioning purposes.

A need arises for techniques that may provide systematic drug repositioning that fully leverages drug-drug interactions.

### SUMMARY

Embodiments of the present systems and methods may provide techniques for systematic drug repositioning that fully leverages drug-drug interactions. In embodiments, the present systems and methods may generate a drug similarity metric based on DDI data and make drug repositioning predictions by leveraging the new similarity metric.

For example, in an embodiment, a computer-implemented method for conducting a drug repositioning trial may comprise receiving data relating to drug-drug interactions, generating drug interaction vectors for each drug in the received data, generating a logistic regression model to generate drug repositioning hypotheses, generating at least one drug repositioning hypothesis using the logistic regression model, and conducting a drug repositioning trial using the generated drug repositioning hypothesis.

In embodiments, the data relating to drug-drug interactions may comprise at least one of data from a drug database, drug labeling information, and data obtained directly from a drug trial. The generated drug interaction vectors may comprise at least one of binary vectors, continuous vectors, and categorical vectors. The generated drug similarity measures may comprise at least one of a Tanimoto coefficient, a cosine similarity, and a Euclidean distance. The method may further comprise generating drug similarity measures for each drug in the received data using the generated drug interaction vectors, generating similarity matrices representing drugs and including the generated drug similarity measures, and generating the logistic regression model using the generated similarity matrices. The generated logistic regression models may be generated using the generated similarity matrices in combination with other drug similarity metrics. The other drug similarity metrics may comprise at least one of chemical structure information and target binding-based metrics.

In an embodiment, a system for conducting a drug repositioning trial may comprise a processor, memory accessible by the processor, and computer program instructions stored in the memory and executable by the processor to perform receiving data relating to drug-drug interactions, generating drug interaction vectors for each drug in the received data, generating a logistic regression model to generate drug repositioning hypotheses, generating at least one drug repositioning hypothesis using the logistic regression model, and conducting a drug repositioning trial using the generated drug repositioning hypothesis.

In an embodiment, a computer program product for conducting a drug repositioning trial may comprise a non-transitory computer readable storage having program instructions embodied therewith, the program instructions executable by a computer, to cause the computer to perform a method comprising receiving data relating to drug-drug interactions, generating drug interaction vectors for each drug in the received data, generating a logistic regression model to generate drug repositioning hypotheses, generating at least one drug repositioning hypothesis using the logistic regression model, and conducting a drug repositioning trial using the generated drug repositioning hypothesis.

### BRIEF DESCRIPTION OF THE DRAWINGS

The details of the present invention, both as to its structure and operation, can best be understood by referring to the accompanying drawings, in which like reference numbers and designations refer to like elements.

FIG. 1 illustrates an exemplary system in which the present systems and methods may be implemented.

FIG. 2 is an exemplary data flow diagram of a process according to embodiments of the present systems and methods.

FIG. 3 is an exemplary block diagram of a computer system in which processes involved in the embodiments described herein may be implemented.

FIG. 4 is an exemplary illustration of a model used to classify a given drug as treating or not-treating a disease.

FIG. 5 is an exemplary illustration of training a supervised classifier for each disease.

### DETAILED DESCRIPTION

Embodiments of the present systems and methods may provide techniques for systematic drug repositioning that fully leverages drug-drug interactions. In embodiments, the present systems and methods may generate a drug similarity metric based on DDI data and make drug repositioning predictions by leveraging the new similarity metric.

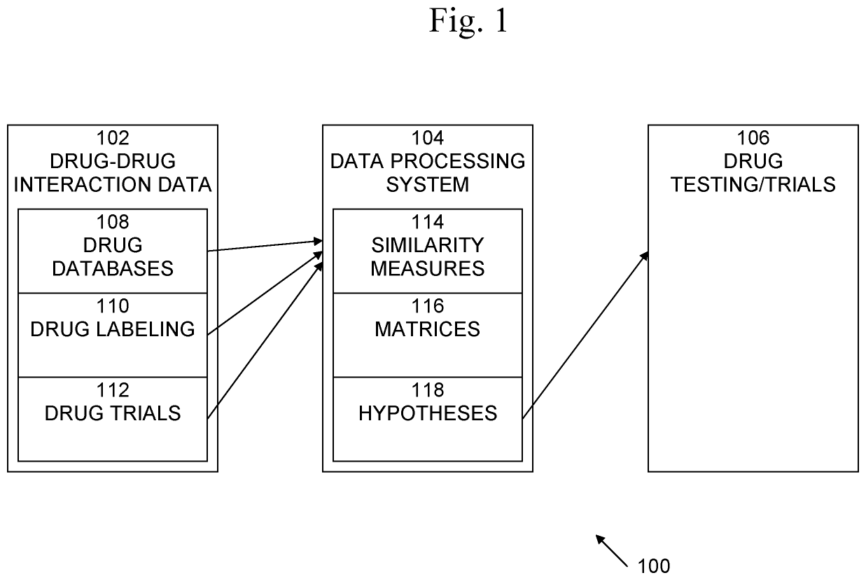

An exemplary system 100 in which the present systems and methods may be implemented is shown in FIG. 1. In this example, system 100 includes drug-drug interaction (DDI) data sources 102, data processing system 104, and drug testing 106. DDI data may include data from drug databases 108, such as the curated DRUGBANK®, MEDI-SPAN®, etc., drug labeling information 110, such as may be obtained from manufacturers, from the FDA Online Label Repository, etc., and data obtained directly from drug trials. Data processing system 104 may include one or more computer systems including software and data to generated similarity measures 114, matrices 116, and drug repositioning hypotheses 118. Drug testing/trials 106 may include experimental or observational research studies on animals or human participants in order to test the validity of the generated drug repositioning hypotheses 118.

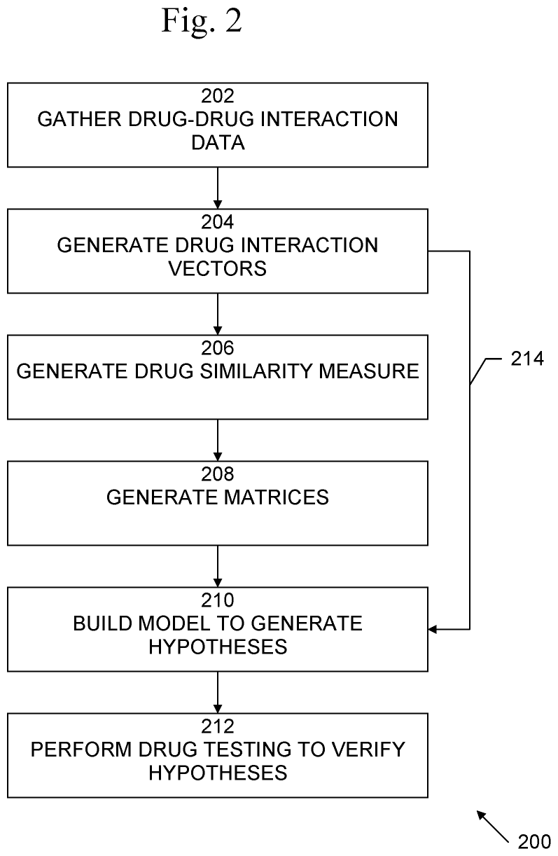

FIG. 2 is a flow diagram of a process 200 which may be implemented in the system of FIG. 1. Process 200 begins with 202, in which DDI data may be obtained from DDI sources 102, shown in FIG. 1. DDI data may include data from drug databases 108, such as the curated DRUGBANK®, MEDI-SPAN®, etc., drug labeling information 110, such as may be obtained from manufacturers, from the FDA Online Label Repository, etc., and data obtained directly from drug trials. At 204, the obtained DDI data may be processed to form drug interaction vectors that may represent the DDI data in a form that may be useful for further processing. For example, DDI data may be represented as binary vectors (whether two drugs interact or not), continuous vectors (by considering level of severalties when DDIs happen), or categorical vectors (by considering different groups of actions caused by DDIs).

At 206, drug similarity measures may be generated using the drug interaction vectors. To generate measures based on DDI information, a number of processing techniques may be used, such as the Tanimoto coefficient, cosine similarity, or Euclidean distance. For example, using binary vectors and the Tanimoto coefficient, each drug may be represented by an N-dimensional binary profile, where N is the number of drugs in the obtained DDI data, the input dataset. Each element of the N-dimensional binary profile may encode for interaction or non-interaction of each drug in the input dataset by 1 or 0, respectively. The Tanimoto coefficient (TC) may then be used to compute similarities among all the DDI profiles. The Tanimoto coefficient between two DDI profiles A and B may be defined as a ratio between the number of features in the intersection to the union of both profiles: TC(A,B)=|A∩B|/|A∪B|. This may, for example, be computed as:

$$\frac{{\sum }_{j = 1}^{k} {a}_{j} \times {b}_{j}}{( {\sum }_{j = 1}^{k} {a}_{j}^{2} + {\sum }_{j = 1}^{k} {b}_{j}^{2} - {\sum }_{j = 1}^{k} {a}_{j} \times {b}_{j} )} .$$

The cosine similarity between two DDI profiles A and B may be defined as

$$\frac{A \cdot B}{‖ A ‖ ‖ B ‖} = \frac{{\sum }_{i = 1}^{n} {A}_{i} {B}_{i}}{\sqrt{{\sum }_{i = 1}^{n} {A}_{i}^{2}} \sqrt{{\sum }_{i = 1}^{n} {B}_{i}^{2}}} .$$

Likewise, the Euclidean distance between two DDI profiles A and B may be defined as

∥A−B∥=\sqrt{(A−B)·(A−B)}=\sqrt{(a₁−b₁)²+(a₂−b₂)²+ . . . +(aₙ−bₙ)²}.

At 208, matrices may be generated so that the rows and columns represent drugs and each cell represents the similarity measure, such as the Tanimoto coefficient, cosine similarity, or Euclidean distance between drugs based on the input dataset. At 210, the resulting similarity matrices may be used alone or in combination with other drug similarity metrics, such as chemical structure or target binding-based metrics, to build features for models, such as large-scale logistic regression models, which may be used to generate drug repositioning hypotheses.

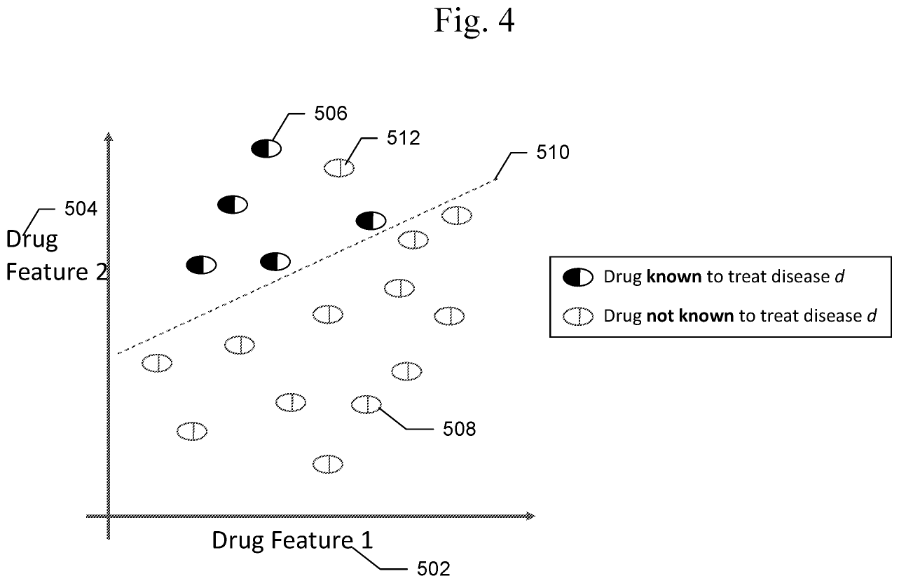

Alternatively, in embodiments, logistic regression models may be built based on 204 to predict 210, as in path 214. For each disease, a separate model or per-disease classifier may be developed to predict whether the drug can treat the disease, as shown in FIG. 4. In the example shown in FIG. 4, Features values (depicted along the x- and y-axis) are given for a set of drugs. For example, drug feature 1 502 may be shown on the x-axis, and drug feature 2 504 may be shown on the y-axis. Some drugs 506 may be known to treat a given disease, while for some drugs 508, it may not be known whether they treat disease d. A model 510, here represented by a line, may be trained to classify a given drug as treating or not-treating the disease. Thus, as shown in this example, a drug 512, that is not known to treat disease d may be predicted by model 510 to be able to treat disease d. Drug 512 may then be tested to determine whether or not it is able to treat disease d.

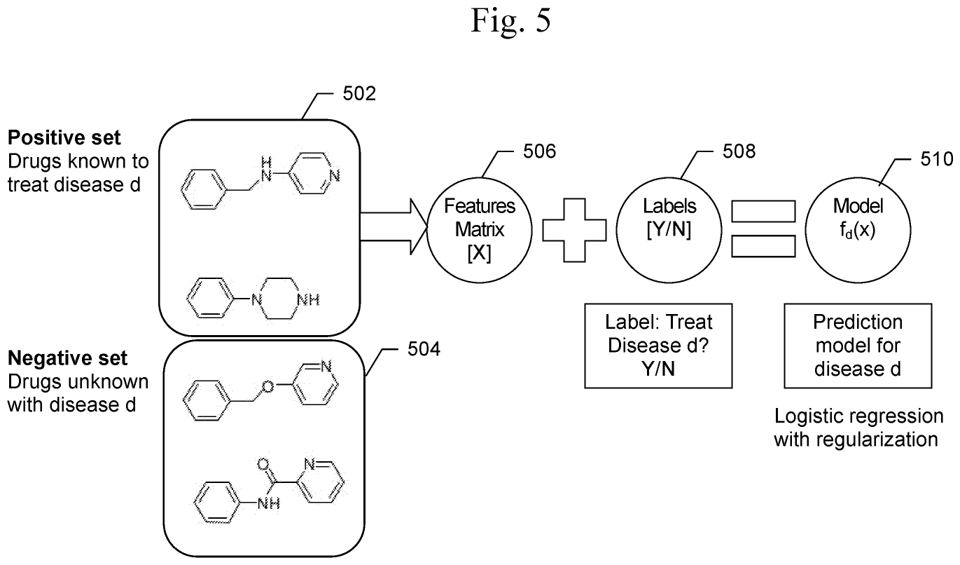

An example of prediction is shown in FIG. 5. In this example, the “positive set” 502 may include drugs that are known to treat disease d and “negative set” 504 may include drugs that are not known to treat disease d. Such knowledge may also be called labels 508. A features matrix 506 may be generated from the positive set 502 and the negative set 504, as in 204 in FIG. 2. A model 510 maybe generated from features matrix 506 and labels 508, as in 210 in FIG. 2. The model may directly utilize the feature vector to make predictions without needing to calculate similarities.

Accordingly, the present systems and methods may leverage DDI-based drug similarity metric as features, and predict clearly-defined drug indication categories. DDI data may be combined with other types of drug knowledge such as chemical structure and target binding to obtain higher prediction accuracy. Alternatively, the present systems and methods may leverage DDI information directly as features, and predict clearly-defined drug indication categories.

For example, based on DDI, classifiers obtained an area under the receiver operating characteristic curve (AUROC) of 0.986 for predicting depression and 0.917 for predicting Parkinson's disease as drug indications. As a comparison, methods based on chemical structure or molecular docking obtained no more than 0.923 for predicting depression and 0.875 for predicting Parkinson's disease as drug indications. The feature analytics function may list similar known drug-indication pairs that may be referred to understand the new predictions. This is because similar drugs may tend to treat similar disease. Such similarity information may be obtained at 206 and 208 in FIG. 2. By leveraging the clinical or phenotypical data from pharmacological knowledgebase, such as drug-drug interactions, the prediction accuracy of drug repositioning hypothesis generation may be improved.

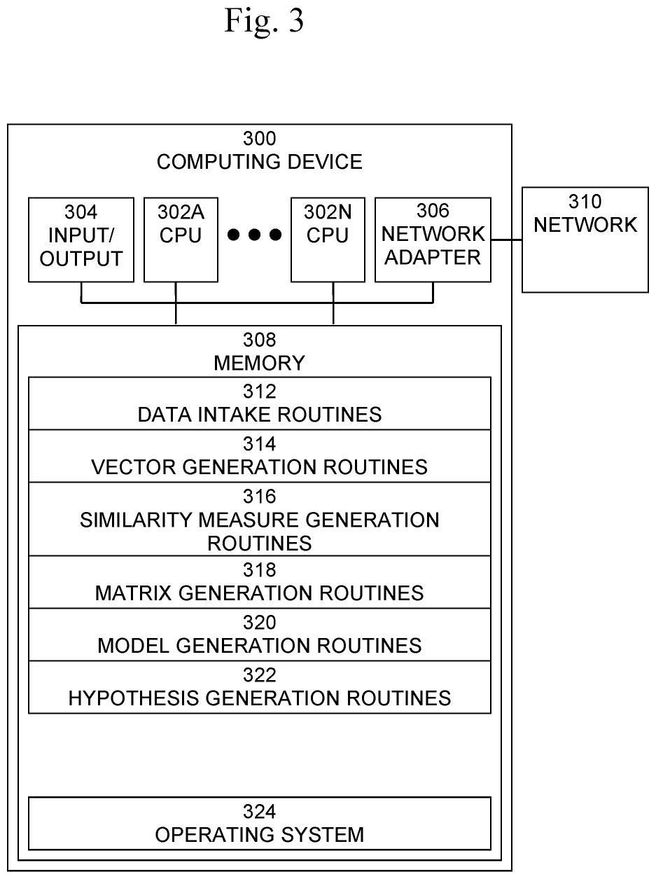

An exemplary block diagram of a computer system 302, in which processes involved in the embodiments described herein may be implemented, is shown in FIG. 3. Computer system 302 may be implemented using one or more programmed general-purpose computer systems, such as embedded processors, systems on a chip, personal computers, workstations, server systems, and minicomputers or mainframe computers, or in distributed, networked computing environments. Computer system 302 may include one or more processors (CPUs) 302A-302N, input/output circuitry 304, network adapter 306, and memory 308. CPUs 302A-302N execute program instructions in order to carry out the functions of the present communications systems and methods. Typically, CPUs 302A-302N are one or more microprocessors, such as an INTEL CORE® processor. FIG. 3 illustrates an embodiment in which computer system 302 is implemented as a single multi-processor computer system, in which multiple processors 302A-302N share system resources, such as memory 308, input/output circuitry 304, and network adapter 306. However, the present communications systems and methods also include embodiments in which computer system 302 is implemented as a plurality of networked computer systems, which may be single-processor computer systems, multi-processor computer systems, or a mix thereof.

Input/output circuitry 304 provides the capability to input data to, or output data from, computer system 302. For example, input/output circuitry may include input devices, such as keyboards, mice, touchpads, trackballs, scanners, analog to digital converters, etc., output devices, such as video adapters, monitors, printers, etc., and input/output devices, such as, modems, etc. Network adapter 306 interfaces device 300 with a network 310. Network 310 may be any public or proprietary LAN or WAN, including, but not limited to the Internet.

Memory 308 stores program instructions that are executed by, and data that are used and processed by, CPU 302 to perform the functions of computer system 302. Memory 308 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

The contents of memory 308 may vary depending upon the function that computer system 302 is programmed to perform. In the example shown in FIG. 3, exemplary memory contents are shown representing routines and data for embodiments of the processes described above. However, one of skill in the art would recognize that these routines, along with the memory contents related to those routines, may not be included on one system or device, but rather may be distributed among a plurality of systems or devices, based on well-known engineering considerations. The present communications systems and methods may include any and all such arrangements.

In embodiments, at least a portion of the software shown in FIG. 3 may be implemented on a current leader server. Likewise, in embodiments, at least a portion of the software shown in FIG. 3 may be implemented on a computer system other than the current leader server.

In the example shown in FIG. 3, memory 308 may include data intake routines 312, vector generation routines 314, similarity measure generation routines 316, matrix generation routines 318, model generation routines 320, hypothesis generation routines 322, and operating system 324. Data intake routines 312 may include software routines to obtain DDI data, for example, from drug databases, drug labeling information, and from drug trials. Vector generation routines 314 may include software routines to generate drug interaction vectors that may represent the DDI data in a form that may be useful for further processing, such as binary vectors, continuous vectors, or categorical vectors. Similarity measure generation routines 316 may include software routines to generate drug similarity measures using the drug interaction vectors. Matrix generation routines 318 may include software routines to generate matrices in which the rows and columns represent drugs and each cell represents the similarity measure. Model generation routines 320 may include software routines to generate models, such as logistic regression models. Hypothesis generation routines 322 may include software routines to generate drug repositioning hypotheses using the models. Operating system 324 may provide overall system functionality.

As shown in FIG. 3, the present communications systems and methods may include implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

What is claimed is:

1. A computer-implemented method for conducting a drug repositioning trial, the method comprising:
receiving data relating to drug-drug interactions;
generating drug interaction vectors for each drug in the received data;
generating a logistic regression model to generate drug repositioning hypotheses;
generating at least one drug repositioning hypothesis using the logistic regression model; and
conducting a drug repositioning trial using the generated drug repositioning hypothesis.

2. The method of claim 1, wherein the data relating to drug-drug interactions comprises at least one of data from a drug database, drug labeling information, and data obtained directly from a drug trial.

3. The method of claim 2, wherein the generated drug interaction vectors comprise at least one of binary vectors, continuous vectors, and categorical vectors.

4. The method of claim 2, wherein the generated drug similarity measures comprise at least one of a Tanimoto coefficient, a cosine similarity, or a Euclidean distance.

5. The method of claim 1, further comprising:
generating drug similarity measures for each drug in the received data using the generated drug interaction vectors;
generating similarity matrices representing drugs and including the generated drug similarity measures; and
generating the logistic regression model using the generated similarity matrices.

6. The method of claim 5, wherein the generated logistic regression models are generated using the generated similarity matrices in combination with other drug similarity metrics.

7. The method of claim 6, wherein the other drug similarity metrics comprise at least one of chemical structure information and target binding based metrics.

8. A system for conducting a drug repositioning trial, the system comprising a processor, memory accessible by the processor, and computer program instructions stored in the memory and executable by the processor to perform:
receiving data relating to drug-drug interactions;
generating drug interaction vectors for each drug in the received data;
generating a logistic regression model to generate drug repositioning hypotheses;
generating at least one drug repositioning hypothesis using the logistic regression model; and
conducting a drug repositioning trial using the generated drug repositioning hypothesis.

9. The system of claim 8, wherein the data relating to drug-drug interactions comprises at least one of data from a drug database, drug labeling information, and data obtained directly from a drug trial.

10. The system of claim 9, wherein the generated drug interaction vectors comprise at least one of binary vectors, continuous vectors, and categorical vectors.

11. The system of claim 9, wherein the generated drug similarity measures comprise at least one of a Tanimoto coefficient, a cosine similarity, or a Euclidean distance.

12. The system of claim 8, further comprising:
generating drug similarity measures for each drug in the received data using the generated drug interaction vectors;
generating similarity matrices representing drugs and including the generated drug similarity measures; and
generating the logistic regression model using the generated similarity matrices.

13. The system of claim 12, wherein the generated logistic regression models are generated using the generated similarity matrices in combination with other drug similarity metrics.

14. The system of claim 13, wherein the other drug similarity metrics comprise at least one of chemical structure information and target binding based metrics.

15. A computer program product for conducting a drug repositioning trial, the computer program product comprising a non-transitory computer readable storage having program instructions embodied therewith, the program instructions executable by a computer, to cause the computer to perform a method comprising:
receiving data relating to drug-drug interactions;
generating drug interaction vectors for each drug in the received data;
generating a logistic regression model to generate drug repositioning hypotheses;
generating at least one drug repositioning hypothesis using the logistic regression model; and
conducting a drug repositioning trial using the generated drug repositioning hypothesis.

16. The computer program product of claim 15, wherein the data relating to drug-drug interactions comprises at least one of data from a drug database, drug labeling information, and data obtained directly from a drug trial.

17. The computer program product of claim 16, wherein the generated drug interaction vectors comprise at least one of binary vectors, continuous vectors, and categorical vectors.

18. The computer program product of claim 16, wherein the generated drug similarity measures comprise at least one of a Tanimoto coefficient, a cosine similarity, or a Euclidean distance.

19. The computer program product of claim 15, further comprising:
generating drug similarity measures for each drug in the received data using the generated drug interaction vectors;
generating similarity matrices representing drugs and including the generated drug similarity measures; and
generating the logistic regression model using the generated similarity matrices.

20. The computer program product of claim 19, wherein the generated logistic regression models are generated using the generated similarity matrices in combination with other drug similarity metrics comprising at least one of chemical structure information and target binding based metrics.
